(19) United States
(12) Patent Application Publication
(10) Pub. No.: US 2008/0027095 A1
(43) Pub. Date: Jan. 31, 2008

(54) Use Of 3-Substituted-2-(Diphenylmethy)-1-Azabicyclo[2.2.2]Octanes For Treating Mrg-X1 Receptor Mediated Diseases

(76) Inventors: Priya Kunapuli, Blue Bell, PA (US); Berta Strulovici, Lower Gwynedd, PA (US)

Correspondence Address: MERCK AND CO., INC, P O BOX 2000, RAHWAY, NJ, 07065-0907, US

(21) Appl. No.: 11/794,400

(22) PCT Filed: Jan. 3, 2006
(86) PCT No.: PCT/US06/00055

§ 371 Date: Jun. 27, 2007

Related U.S. Application Data
(60) Provisional application No. 60/642,230, filed on Jan. 7, 2005.

(51) Int. Cl. A61K 31/439; A61P 25/04; G01N 33/00

(52) U.S. Cl. 514/305; 436/96

(57) ABSTRACT

The invention encompasses a method for treating a disease or condition mediated by the human MRG-X1 receptor, such as nociception, hyperalgesia, allodynia, pain related to central hypersensitivity conditions, somatic pain, visceral pain, acute pain, chronic pain, post-operative pain, headache, inflammatory pain, neurological pain, musculoskeletal pain, cancer related pain or vascular pain, in a human patient in need thereof comprising administering to the patient a therapeutically effective amount of a 3-substituted-2-(diphenylmethy)-1-azabicyclo[2.2.2]octane or a pharmaceutically acceptable salt thereof. The invention is also directed to the use of these compounds as molecular tools to directly explore the role of the MRG-X1 receptor in pain perception.

### BACKGROUND OF THE INVENTION

G protein-coupled receptors (GPCRs) constitute one of the largest family of druggable targets in the human genome (1). Not surprisingly, approximately 45% of currently available pharmaceutical drugs are targeted against this class of cell surface receptors (2, 3). The vast genomics effort has led to the identification of a number of GPCRs in the human genome, some of which remain “orphan” with unknown endogenous ligand and function (4, 5). In this post-genomic era, a considerable research effort is aimed at de-orphanizing these receptors and understanding their role in human physiology and their potential therapeutic value.

The MRG family of receptors are one such family of recently identified orphan GPCRs named after the mas-related-genes (6), also called the SNSR for sensory neuron-specific receptors (7). This large gene family consists of 32 murine and 4 human genes (hMRG-X1-hMRG-X4). The specific expression of these receptors in sensory neurons of the dorsal root ganglion (DRG), in addition to sharing a common precursor with the opioidergic nociceptive system, implicates a possible role for the MRG receptors in nociception (6,7). Among the MRG receptors, the hMRG-X1 (also called SNSR3) is expressed solely in the DRG neurons (6). However, further validation of this molecular target in pain perception is hampered by the lack of specific receptor antagonists that could be used as molecular tools for exploring and validating the physiological role of such newly identified molecular targets. The MRG-X1 receptor has been shown to be activated by the proenkaphlin product, BAM22 (7). Interestingly, BAM22 exhibits the classical opioid YGGFM (Met-enkephalin) motif at the N-terminus, and as expected, binds to opioid and MRG receptors (8,9). However, the MRG receptor activity resides in the C-terminal 15 aminoacids of BAM22 (called BAM15) while the N-terminal Met-enkephalin motif within the first 8 amino acids of BAM22 is required for opioid receptor activity (7) and is dispensable for the MRG receptor activity. Furthermore, the MRG receptors are insensitive to the classical opioid receptor antagonists. Thus, the opioid and MRG receptors exhibit potentially similar physiological roles (nociception) in spite of distinct structure-activity relationships and pharmacology with known ligands.

To further understand the physiological role of the MRG receptors, we attempted to expand the repertoire of available molecular tools to study the MRG-X1 receptor by identifying specific receptor antagonists. Since the MRG-X1 receptors have previously been shown to couple to the release of intracellular Ca²⁺ (i[Ca²⁺]) upon activation, (6,7) we took advantage of this prior knowledge to develop a stable beta-lactamase (BLA) reporter-gene cell line in CHO cells, wherein the agonist-induced i[Ca²⁺] response, presumably via the activation of Gq and PLC, would be linked through the calcineurin-NFAT pathway (10) to the BLA reporter under the control of the NFAT promoter (11,12). After clonal selection of one such functional cell by fluorescence-activated cell sorting (FACS), the expanded population of clonal cells was used for ultra high throughput screening of a library of ˜1 million synthetic small molecule compounds in a 1.8 μl cell-based reporter-gene BLA assay. This strategy led to the identification of several classes of hMRG-X1 antagonists and were confirmed by a i[Ca²⁺] transient assay and a receptor binding assay. The mechanism of action of some of these compounds was explored further by the high content receptor internalization assay.

The present invention is directed to the use 3-substituted-2-(diphenylmethy)-1-azabicyclo[2.2.2]octanes as human MRG-X1 receptor antagonists. This class of compounds has previously been described as tachykinin antagonists in U.S. Pat. No. 5,242,930, granted Sep. 7, 1993, and U.S. Pat. No. 5,256,671, granted Oct. 26, 1993. The invention is directed to the use of these compounds as antagonists of the MRG-X1 receptor for treating diseases or conditions mediated by this receptor. The invention is also directed to the use of these compounds as molecular tools to directly explore the role of the MRG-X1 receptor in pain perception.

### SUMMARY OF THE INVENTION

The invention encompasses a method for treating a disease or condition mediated by the human MRG-X1 receptor, such as nociception, hyperalgesia, allodynia, pain related to central hypersensitivity conditions, somatic pain, visceral pain, acute pain, chronic pain, post-operative pain, headache, inflammatory pain, neurological pain, musculoskeletal pain, cancer related pain or vascular pain, in a human patient in need thereof comprising administering to the patient a therapeutically effective amount of a 3-substituted-2-(diphenylmethy)-1-azabicyclo[2.2.2]octane or a pharmaceutically acceptable salt thereof. The invention is also directed to the use of these compounds as molecular tools to directly explore the role of the MRG-X1 receptor in pain perception.

### BRIEF DESCRIPTION OF THE DRAWINGS

FIG. 1. FACS analysis of CHO-hMRG-X1-NFAT/BLA cells: Dot plots of fluorescence intensities of the CCF₄ FRET indicator of BLA activity in CHO-hMRGX1-NFAT/BLA cells as measured by FACSVantage SE flow cytometer using FL4-530/30 and FL5-450/30 detectors excited by 409 nm krypton laser. (A), CHO-hMRG-X1/NFAT-Bla bulk transfected cells were loaded with CCF₄/AM or (B) stimulated with 50 nM BAM15 for 4 hours at 37° C. and then loaded with CCF4/AM, and then subjected to FACS as described in “Experimental Procedures”. Cells in quadrant I, II, III, and IV represent cells emitting blue fluorescence, cells emitting blue/green fluorescence, cells emitting green fluorescence and dead cells, respectively.

FIG. 2. Pharmacological characterization of CHO-hMRG-X1-NFAT/BLA clones: (A), BAM15-induced i[Ca²⁺] in CHO-hMRG-X1/NFAT-Bla cells. Cells were plated in 384-well plates at a density of 8000 cells/well, loaded with 8 μM Fluo-4 AM for one hour at 37 C, and stimulated with BAM15 as detailed in “Experimental Procedures”. Data presented are representative of the 2 independent experiments performed with 4 replicates. (B), BAM15-induced β-lactamase formation in CHO-hMRG-X1/NFAT-Bla cells. Cells were plated at a density of 6000 cells/well in 25 μl of growth medium. Cells were stimulated the next day in 45 μl of assay medium with 5 μl of 10X stock of BAM15 (dilutions made in assay medium: IMDM, 25 mM Hepes, 0.1% BSA) for 4 hours at 37° C., followed by the addition of 10 μl of 6×CCF₄/AM substrate loading buffer for one hour at room temperature. Plates were analyzed on Tecan Spectrofluorplus microplate reader as described in “Experimental Procedures”. Data presented are representative of 2 independent experiments performed with 4-8 replicates.

FIG. 3. Miniaturization on the hMRG-X1 beta-lactamase assay into 3456-well 2 ul assay format. (A) The CHO-hMRGX1-NFAT/BLA cells were plated at a density of 3000 cells/well in 1.4 ul assay medium and stimulated with 0.4 μl of varying concentrations of BAM15 for 4 hours at 37° C., followed by the addition of 0.4 μl of the 6XCCF4 dye mix for one hour at room temperature and reading the assay plate on the tcPR (Excitation: 405 nm; emission: 460 nm, 530 nm), (B) A whole assay plate from the MRGX1 primary screen, showing controls, compounds and hits (actives). The 3× standard deviation for this assay plate was 18.5% inhibition, with a Signal/Basal of 4.9, and a Coefficient of Variance of 6.7%. (C) Percent coefficient of variance for every assay plate in HTS, indicating the 3× standard deviation for the entire screen (18.5% inhibition).

FIG. 4. Agonist-induced hMRG-X1 receptor internalization. U2OS-hMRGX1-βarrGFP cells were plated at a density of 3000 cells/well in 20 μl growth medium and stimulated with varying concentrations of BAM15 for 30 minutes at 37° C. Cells were fixed with paraformaldehyde, nuclei stained with DRAQ5 and visualized under confocal microscopy as described in “Experimental Procedures”. A representative BAM15-induced dose response in N grains is plotted as an average of triplicate wells.

FIG. 5. MRG-X1 receptor antagonists in the receptor trafficking assay. An example of a competitive receptor antagonist and a non-competitive compound in the hMRGX1 receptor internalization assay. (A): images from InCell 300 confocal microscopy, (B): dose response curves for receptor internalization and compound toxicity.

### DETAILED DESCRIPTION OF THE INVENTION

The invention encompasses a method for treating a disease or condition mediated by the human MRG-X1 receptor in a human patient in need thereof comprising administering to the patient a therapeutically effective amount of a compound of Formula I 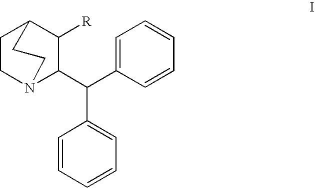
or a pharmaceutically acceptable salt thereof, wherein:
R is 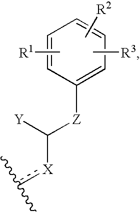
wherein: [symbol]
represents an optional double bond;
when [symbol] is a single bond, X is selected from the group consisting of: —O—, —S—, —NH— and CH₂—;
when [symbol] is a double bond, X is selected from the group consisting of: ═N— and ═CH—;
Y is selected from the group consisting of: H, —OH, ═O, ═S and halo;
Z is selected from the group consisting of: a bond, —O—, —S—, —NH— and —CH₂—;
R¹, R² and R³ are independently selected from the group consisting of: H, C₁₋₆alkyl, C₂₋₆alkenyl, C₂₋₆alkynyl, halo, cyano, nitro, trifluoromethyl, trimethylsilyl, —OR^{a}, SR^{a}, SOR^{a}, SO₂R^{a}, —NR^{a}R^{b}, —NR^{a}COR^{b}, —NR^{a}CO₂R^{b}, —CO₂R^{a} and —CONR^{a}R^{b},
and any two of R¹, R² or R³ may be joined together with the phenyl atom to which they are attached to form naphthyl; and
R^{a} and R^{b} are independently selected from the group consisting of: H, C₁₋₆alkyl, phenyl and trifluoromethyl.

Within this genus, the invention encompasses the method of using a subgenus of compounds wherein Z is —NH—.

Also within this genus, the invention encompasses the method of using a subgenus of compounds wherein [symbol] is a bond.

Also within this genus, the invention encompasses the method of using a subgenus of compounds wherein [symbol] represents a double bond.

Also within this genus, the invention encompasses the method of using a subgenus of compounds wherein [symbol] represents a single bond.

Also within this genus, the invention encompasses the method of using a subgenus of compounds wherein X is O.

Also within this genus, the invention encompasses the method of using a subgenus of compounds wherein Y is OH.

Also within this genus, the invention encompasses the method of using a subgenus of compounds wherein Y is ═O.

Also within this genus, the invention encompasses the method of using a subgenus of compounds wherein R is selected from the following table: 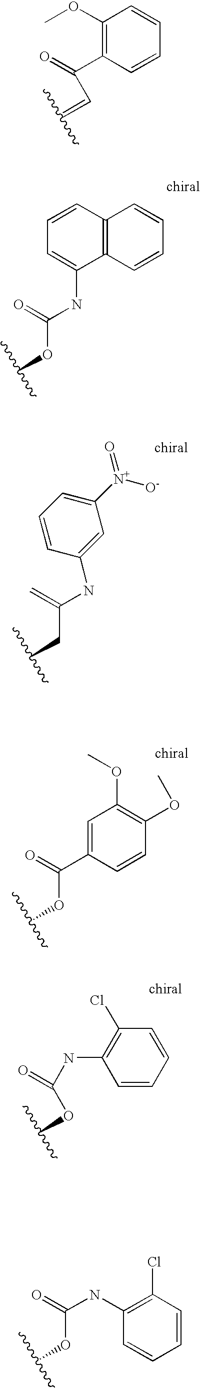 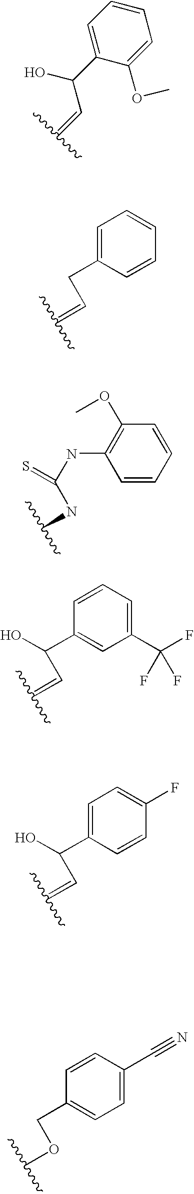
or a pharmaceutically acceptable salt of any of the above compounds.

The invention also encompasses a method for treating a disease or condition mediated by the human MRG-X1 receptor in a human patient in need thereof comprising administering to the patient a therapeutically effective amount of a compound of Formula I 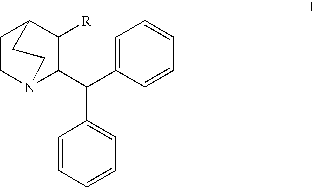
or a pharmaceutically acceptable salt thereof, wherein:
R is 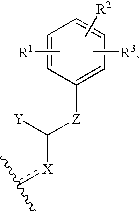
wherein: [symbol]
represents an optional double bond;
when [symbol] is a single bond, X is selected from the group consisting of: —O—, —S—, —NH— and —CH₂—;
when [symbol] is a double bond, X is selected from the group consisting of: ═N— and ═CH—;
Y is selected from the group consisting of: H, —OH, ═O, ═S and halo;
Z is selected from the group consisting of: a bond, —O—, —S—, —NH— and —CH₂—;
R¹, R² and R³ are independently selected from the group consisting of: H, C₁₋₆alkyl, C₂₋₆alkenyl, C₂₋₆alkynyl, halo, cyano, nitro, trifluoromethyl, trimethylsilyl, —OR^{a}, SR^{a}, SOR^{a}, SO₂R^{a}, —NR^{a}R^{b}, —NR^{a}COR^{b}, —NR^{a}CO₂R^{b}, —CO₂R^{a} and —CONR^{a}R^{b},
and any two of R¹, R² or R³ may be joined together with the phenyl atom to which they are attached to form naphthyl; and
R^{a} and R^{b} are independently selected from the group consisting of: H, C₁₋₁₆alkyl, phenyl and trifluoromethyl;
wherein the disease or condition mediated by the human MRG-X1 receptor is selected from the group consisting of: nociception, hyperalgesia, allodynia, pain related to central hypersensitivity conditions, somatic pain, visceral pain, acute pain, chronic pain, post-operative pain, headache, inflammatory pain, neurological pain, musculoskeletal pain, cancer related pain and vascular pain.

The invention also encompasses a method for assessing the potency of a candidate compound that is an antagonist of the MRG-X1 receptor comprising:

(1) determining the potency of the candidate compound in a MRG-X1 receptor binding assay;

(2) determining the potency of a compound of Formula I 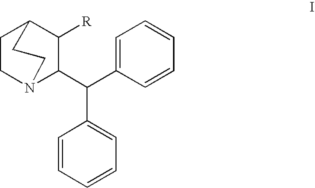
or a pharmaceutically acceptable salt thereof, wherein:
R is 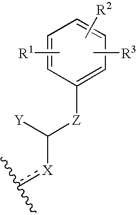
wherein: [symbol] represents an optional double bond;
when is [symbol] a single bond, X is selected from the group consisting of: —O—, —S—, —NH— and —CH₂—;
when [symbol] is a double bond, X is selected from the group consisting of: ═N— and ═CH—;
Y is selected from the group consisting of: H, —OH, ═O, ═S and halo;
Z is selected from the group consisting of: a bond, —, —S—, —NH— and —CH₂—;
R¹, R² and R³ are independently selected from the group consisting of: H, C₁₋₆alkyl, C₂₋₆alkenyl, C₂₋₆alkynyl, halo, cyano, nitro, trifluoromethyl, trimethylsilyl, —OR^{a}, SR^{a}, SOR^{a}, SO₂R^{a}, —NR^{a}R^{b}, —NR^{a}COR^{b}, —NR^{a}CO₂R^{b}, —CO₂R^{a} and —CONR^{a}R^{b},
and any two of R¹, R² or R³ may be joined together with the phenyl atom to which they are attached to form naphthyl; and
R^{a} and R^{b} are independently selected from the group consisting of: H, C₁₋₆alkyl, phenyl and trifluoromethyl, in a MRG-X1 receptor binding assay; and

(3) comparing the potency of the candidate compound to the potency of the compound of Formula I to determine if the candidate is more or less potent than the compound of Formula I. Methods for determining the potency of a compound in a MRG-X1 binding assay are well known in the art an can be accomplished for example by performing dose response studies and determining the IC₅₀.

The invention also encompasses a method for validating the MRG-X1 receptor to treat a disease or condition believed to be mediated by the MRG-X1 receptor, comprising administering an effective amount of a compound of Formula I 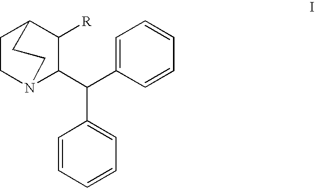
or a pharmaceutically acceptable salt thereof, wherein:
R is 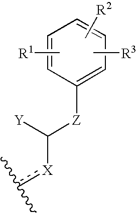
wherein: [symbol]
represents an optional double bond;
when [symbol] is a single bond, X is selected from the group consisting of: —O—, —S—, —NH— and —CH₂—;
when [symbol] is a double bond, X is selected from the group consisting of: ═N— and ═CH—;
Y is selected from the group consisting of: H, —OH, ═O, ═S and halo;
Z is selected from the group consisting of: a bond, —O—, —S—, —NH— and —CH₂—;
R¹, R² and R³ are independently selected from the group consisting of: H, C₁₋₆alkyl, C₂₋₆alkenyl, C₂₋₆alkynyl, halo, cyano, nitro, trifluoromethyl, trimethylsilyl, —OR^{a}, SR^{a}, SOR^{a}, SO₂R^{a}, —NR^{a}R^{b}, —NR^{a}COR^{b}, —NR^{a}CO₂R^{b}, —CO₂R^{a} and —CONR^{a}R^{b},
and any two of R¹, R² or R³ may be joined together with the phenyl atom to which they are attached to form naphthyl; and
R^{a} and R^{b} are independently selected from the group consisting of: H, C₁₋₆alkyl, phenyl and trifluoromethyl, to an animal in an in vivo model to test whether the compound is useful to treat the disease or condition. Within this embodiment the invention encompasses the aforementioned method wherein the disease or condition is pain. Many such in vivo animal models for testing whether a compound is useful to treat a particular disease or condition are known in the art. Animal models for pain are described, for example, in Animal Models of Pain, ILAR Journal, vol. 40, no. 3, 1999.

The invention also encompasses the use of a compound of Formula I 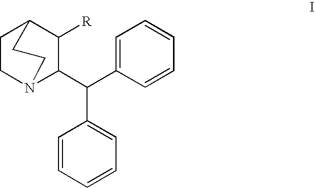
or a pharmaceutically acceptable salt thereof, wherein:
R is 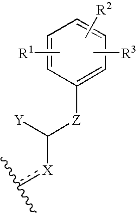
wherein: [symbol]
represents an optional double bond;
when [symbol] is a single bond, X is selected from the group consisting of: —O—, —S—, —NH— and —CH₂—;
when [symbol] is a double bond, X is selected from the group consisting of: ═N— and ═CH—;
Y is selected from the group consisting of: H, —OH, ═O, ═S and halo;
Z is selected from the group consisting of: a bond, —O—, —S—, —NH— and —CH₂—;
R¹, R² and R³ are independently selected from the group consisting of: H, C₁₋₆alkyl, C₂₋₆alkenyl, C₂₋₆alkynyl, halo, cyano, nitro, trifluoromethyl, trimethylsilyl, —OR^{a}, SR^{a}, SOR^{a}, SO₂R^{a}, —NR^{a}R^{b}, —NR^{a}COR^{b}, —NR^{a}CO₂R^{b}, —CO₂R^{a} and —CONR^{a}R^{b},
and any two of R¹, R² or R³ may be joined together with the phenyl atom to which they are attached to form naphthyl; and
R^{a} and R^{b} are independently selected from the group consisting of: H, C₁₋₆alkyl, phenyl and trifluoromethyl, to bind to the MRG-X1 receptor in an in vitro or in vivo assay, test or model.

The compounds of the invention are believed to be useful for treating or preventing pain. In the context of the invention, the term pain can mean nociception, hyperalgesia, allodynia, pain related to central hypersensitivity conditions, somatic pain, visceral pain, acute pain, chronic pain, post-operative pain, headache, inflammatory pain, neurological pain, musculoskeletal pain, cancer related pain or vascular pain. Examples of acute pain include post-operative pain, migraine, headache and trigeminal neuralgia. Examples of chronic pain include pain associated with musculoskeletal disorders such as rheumatoid arthritis, osteoarthritis, ankylosing spondylitis, sero-negative (non-rheumatoid) arthropathies, non-articular rheumatism and peri-articular disorders, and pain associated with cancer, peripheral neuropathy and post-herpetic neuralgia. Examples of pain with an inflammatory component (in addition to some of those described above) include rheumatic pain, dental pain and dysmenorrhoea.

“Pain” is a sensory experience perceived by nerve tissue distinct from sensations of touch, pressure, heat and cold. The range of pain sensations, as well as the variation of perception of pain by individuals, renders a precise definition of pain near impossible. In the context of the present invention, “pain” is used in the broadest possible sense and includes nociceptive pain, such as pain related to tissue damage and inflammation, pain related to noxious stimuli, acute pain, chronic pain, and neuropathic pain.

“Acute pain” is often short-lived with a specific cause and purpose; generally produces no persistent psychological reactions. Acute pain can occur during soft tissue injury, and with infection and inflammation. It can be modulated and removed by treating its cause and through combined strategies using analgesics to treat the pain and antibiotics to treat the infection.

“Chronic pain” is distinctly different from and more complex than acute pain. Chronic pain has no time limit, often has no apparent cause and serves no apparent biological purpose. Chronic pain can trigger multiple psychological problems that confound both patient and health care provider, leading to feelings of helplessness and hopelessness. The most common causes of chronic pain include low-back pain, headache, recurrent facial pain, pain associated with cancer and arthritis pain.

The pain is termed “neuropathic” when it is taken to represent neurologic dysfunction. “Neuropathic pain” has a complex and variable etiology. It is typically characterized by hyperalgesia (lowered pain threshold and enhanced pain perception) and by allodynia (pain from innocuous mechanical or thermal stimuli). Neuropathic pain is usually chronic and tends not to respond to the same drugs as “normal pain” (nociceptive pain), therefore, its treatment is much more difficult. Neuropathic pain may develop whenever nerves are damaged, by trauma, by disease such as diabetes, herpes zoster, or late-stage cancer, or by chemical injury (e.g., as an untoward consequence of agents including the false-nucleotide anti-HIV drugs). It may also develop after amputation (including mastectomy). Examples of neuropathic pain include monoradiculopathies, trigerninal neuralgia, postherpetic neuralgia, complex regional pain syndromes and the various peripheral neuropathies. This is in contrast with “normal pain” or “nociceptive pain,” which includes normal post-operative pain, pain associated with trauma, and chronic pain of arthritis.

“Peripheral neuropathy” is a neurodegenerative disorder that affects the peripheral nerves, most often manifested as one or a combination of motor, sensory, sensorimotor, or autonomic dysfunction. Peripheral neuropathies may, for example, be characterized by the degeneration of peripheral sensory neurons, which may result from a disease or disorder such as diabetes (diabetic neuropathy), alcoholism and acquired immunodeficiency syndrome (AIDS), from therapy such as cytostatic drug therapy in cancer, or from genetic predisposition. Genetically acquired peripheral neuropathies include, for example, Krabbe's disease, Metachromatic leukodystrophy, and Charcot-Marie Tooth (CMT) Disease. Peripheral neuropathies are often accompanied by pain.

Processes for synthesizing the compounds used in the present invention are known in the art, as illustrated in U.S. Pat. No. 5,242,930, granted Sep. 7, 1993, U.S. Pat. No. 5,256,671, granted Oct. 26, 1993, Swain, et al., Journal of Medicinal Chemistry, vol. 38, pp. 4792-4805, 1995, Seward et al., Bioorganic & Medicinal Chemistry Letters, vol. 3, pp. 1361-1366, 1993, and Swain et al., Bioorganic & Medicinal Chemistry Letters, vol. 3, pp. 1703-1706, 1993. Dosage amounts in which to administer these compounds are also known in the art and described in U.S. Pat. No. 5,242,930, granted Sep. 7, 1993 and U.S. Pat. No. 5,256,671, granted Oct. 26, 1993.

### EXAMPLE A

### Materials

All cell culture and molecular biology reagents, Zeocin and CCF₄/AM substrate and Lipofectamine 2000 were purchased from Invitrogen (Carlsbad, Calif.). The beta-lactamase gene reporter plasmids under the control of the NFAT promoter was licensed and obtained from Aurora Biosciences (San Diego, Calif.). The 96- and 384-well black clear bottom tissue culture treated assay plates were purchased from Corning Inc. (Acton, Mass.). The 3456-well black, clear bottom tissue culture treated plates were purchased from Greiner Bio-One (Frickenhausen, Germany). FLIPR and assay reagents for the i[Ca²⁺] assay were purchased from Molecular Devices (Sunnyvale, Calif.). BAM15 peptide (VGRPEWWMDYQKRYG) was custom synthesized by SynPep (Dublin, Calif.). A 10 mM stock solution of BAM15 was made in 0.1N acetic acid and stored at −80° C. until use. BAM15 dilutions were made in aqueous assay medium (IMDM, 25 mM Hepes, 0.1% BSA). For ligand binding experiments, [³H]-BAM was custom synthesized by Amersham/GE Healthcare (Piscataway, N.J.) with a specific activity of 34 Ci/mmol. UltimaGold scintillation solution was purchased from PerkinElmer (Shelton, Conn.). U2OS cells stably expressing mRGX1 receptor and Parrestin-GFP were purchased from Norak Biosciences, Inc. (Morrisville, N.C.). DRAQ5 nuclear stain was purchased from Biostatus Limited (Leicestershire, UK) at a stock concentration of 5 mM.

### EXAMPLE B

### Cell Culture and Transfection

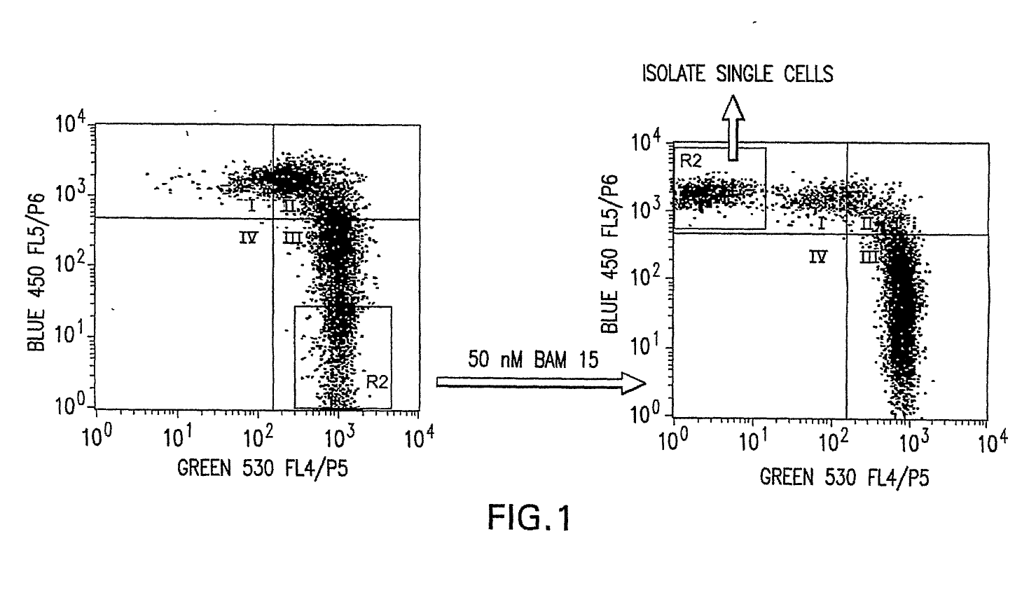

The hMRG-X1 cDNA, cloned into the EcoRI/(SalI/XhoI) site of pcDNA3.1, was transfected into CHO-NFAT-BLA cells stably expressing the NFAT-BlaX reporter (11) using Lipefectamine 2000. Cells were grown in DMEM with 10% serum, 2 mM L-glutamine, 1 mM Non-essential amino acids, 1 mM Sodium pyruvate 25 mM Hepes, pH 7.4, 55 μM 2-mercaptoethanol, 250 μg/ml Zeocin and stable cells were selected by resistance to 1 mg/ml geneticin. CHO-hMRG-X1/NFAT-Bla stable cells exhibiting agonist-induced functional response were clonally selected by FACS analysis as described previously (11) with minor modifications. Briefly, cells exhibiting endogenous signaling in the absence of agonist were first eliminated from the transfected pool of cells by FACS. This was followed by a second round of FACS, after stimulating the cells with 50 nM BAM15 for 4 hours. At this stage, single cells exhibiting maximum blue fluorescence emission at 460 nm (R2 box; 11% of the total population; FIG. 1, right) were collected directly into 200 μl of complete medium in a 96-well plate. These single cell clones were allowed to grow for 3 weeks, and then subjected to more detailed pharmacological characterization.

### EXAMPLE C

### Beta-Lactamase Assay

BLA assays in 384-well plates were performed essentially as described previously (11). For miniaturization of the BLA assay into 3456-well format, cells were serum-starved for ˜18 hours in assay medium (11) the day before the assay. On the day of the assay, cells were dissociated with enzyme-free dissociation buffer, re-suspended into assay medium and dispensed into a 3456-well nanoplate (3000 cells in 1.4 μl/well) using the FRD (Aurora Discovery, San Diego, Calif.) (13). Rest of the BLA assay was conducted essentially as described earlier (13). The cellular response was also observed by fluorescence microscopy with UV illumination. The data are plotted as a ratio of the emissions 460 nm/530 nm. Experimental data points are represented as median±standard deviation of 4-115 replicates.

### EXAMPLE D

### Intracellular Ca²⁺ Measurements

Intracellular Ca²⁺ measurements were performed essentially as described (14, 15). Modifications include 8000 cells/well of the CHO-hMRG-X1-NFAT/Bla cells in 20 μgrowth medium plated in 384-well plates 24 hours before assay. Cells were loaded with 20 μl of 2X calcium probe and fluorescent quencher. Agonist was prepared as a 4× stock in HBSS buffer and added to the assay plate by the FLIPR384 pipetter (13.3 μl/well). Experimental data points are represented as median±standard deviation of 4 replicates.

### EXAMPLE E

### Receptor Trafficking Assay

U2OS-hMRGX1-βarrGFP stable cells were cultured in MEM supplemented with 10% heat-inactivated FBS, 4 mM L-Glutamine, 10 μg/ml Gentamicin, 10 mM HEPES, 0.4 mg/ml G-418, and 0.4 mg/ml Zeocin. Cells were plated on black/clear plastic bottom 384-well Corning plates at a density of 3000 cells/well in 20 μl growth medium and cultured overnight. DMSO, or sample compounds titrated in 75% DMSO were transferred from the compound source plate into the assay plate (200 nl/well). After 15 minutes incubation at 37° C. and 5% CO₂, 5 μl of agonist (varying concentrations of BAM15 or EC80 concentration of 60 μM for compound titrations) was added and cells were incubated at 37° C. for 30 minutes. The fixing/nuclear staining solution was prepared in PBS as a 2× stock containing 4% paraformaldehyde and 2 μM DRAQ5 and 25 μl of this solution was added to the cells and incubated at room temperature in the dark for 30 minutes. The solution was then aspirated and replaced with PBS before confocal microscopy. Confocal microscopy was performed on the IN Cell Analyzer 3000 (GE Healthcare, Piscataway, N.J.).

### EXAMPLE F

### Receptor Binding Assay

CHO-hMRG-X1-NFAT-BLA cells were plated in a 24-well plate at 60,000 cells/well in 500 μl growth medium and incubated at 37° C. for 24 hours. The cells were washed once with 200 μl assay buffer (HANKS with 0.1% BSA and 20 mM HEPES, pH 7.5, refrigerated at 4° C.) using a vacuum aspirator. 90 μl assay buffer was added to each well followed by an addition of 0.2 μl/well diluted compounds (500X stock in DMSO) and incubated at 4° C. for 5 minutes. 10 ml of ³H-BAM peptide was added to each well (10 nM final concentration; ˜75,000 dpm) and the assay plate was incubated at 4° C. for 30 minutes with brief gentle shaking every 5 minutes. The assay plate was then washed rapidly with 2X 200 μl assay buffer (approximately 4 to 5 seconds for both washes). All wash steps were performed in a cold room (4° C.). To lyse the cells, 200 μl of 0.2 M NaOH was added to each well and incubate at room temperature for 30 minutes. The cell lysate in each well was transferred to a scintillation vial followed by addition of 3 ml scintillation solution (UltimaGold, PerkinElmer) and counted in a scintillation counter (1 minute per vial).

### EXAMPLE G

### Data Analysis

All experiments with dose-response relationships were analyzed by a nonlinear regression software package from Graphpad (San Diego, Calif.). In the InCell 3000 imager, two excitation lines, 488 nm and 633 nm, were used to simultaneously excite βarrestin-GFP and the DRAQ5 nuclear stain, respectively. Confocal images from the IcCell 3000 were analyzed by the Raven software. Images of 200-300 cells from each well were captured and analyzed with GRN1 (granularity) algorithm using the Raven software of IN Cell Analyzer 3000. Individual cells or nuclei are first identified by thresholding the red channel (DRAQ5). From the nucleus, a rectangular bounding box was dilated out to the edge of the cell with a dilation setting of 15 pixels. Within this bounding box, fluorescent spots of βarrestin-GFP distribution were identified and outlined using an intensity gradient of 1.2 and grain size of 4 pixels. This granularity algorithm was used to measure the number of fluorescent spots (Ngrains) based on fluorescent intensity and size of the grains and the average value from all the cells in a well were used to obtain the “Ngrain” value for the well. The same GRN1 algorithm was used to measure toxic affects or other false positives. Icyt value was used to measure the average intensity of the green fluorescence inside the bounding box as described above. Low Icyt values may indicate a sign of toxicity (resulting in either cell lyses or cells rounding up) displaying little or no GFP distribution in the cytoplasm and the plasma membrane, while high Icyt values may be due to the presence of fluorescent compound.

Results and Discussion

Clonal Selection and Pharmacological Characterization

Since the hMRG-X1 receptors were previously shown to activate i[Ca²⁺] upon agonist stimulation (6, 7), we developed a BLA reporter-gene assay for the hMRG-X1 receptors in CHO cells, such that agonist stimulation in these cells would activate the Gq-PLC-Ca²⁺-calcineurin-NFAT-BLA pathway (10, 11). To increase the chances of finding hMRG-X1 receptor antagonists, we chose to isolate pure, single cell clones exhibiting high sensitivity to the agonist in the BLA assay by FACS. FIG. 1 shows a histogram of the transfected pool of cells, wherein, a small subset of the cells (R2, 17% of total population in FIG. 1, left) exhibited high green (530 nm) and low blue (460 nm) fluorescence emission when excited at 405 nm due to FRET within the CCF4 substrate in the absence of agonist stimulation, representing cells with minimal endogenous signaling. A subset of these cells also exhibited appropriate agonist-induced FRET response, as observed after stimulation with submaximal 50 nW BAM15 (R2, 11% of total population in FIG. 1, right). Single cell clones from this population were analyzed further for appropriate receptor pharmacology.

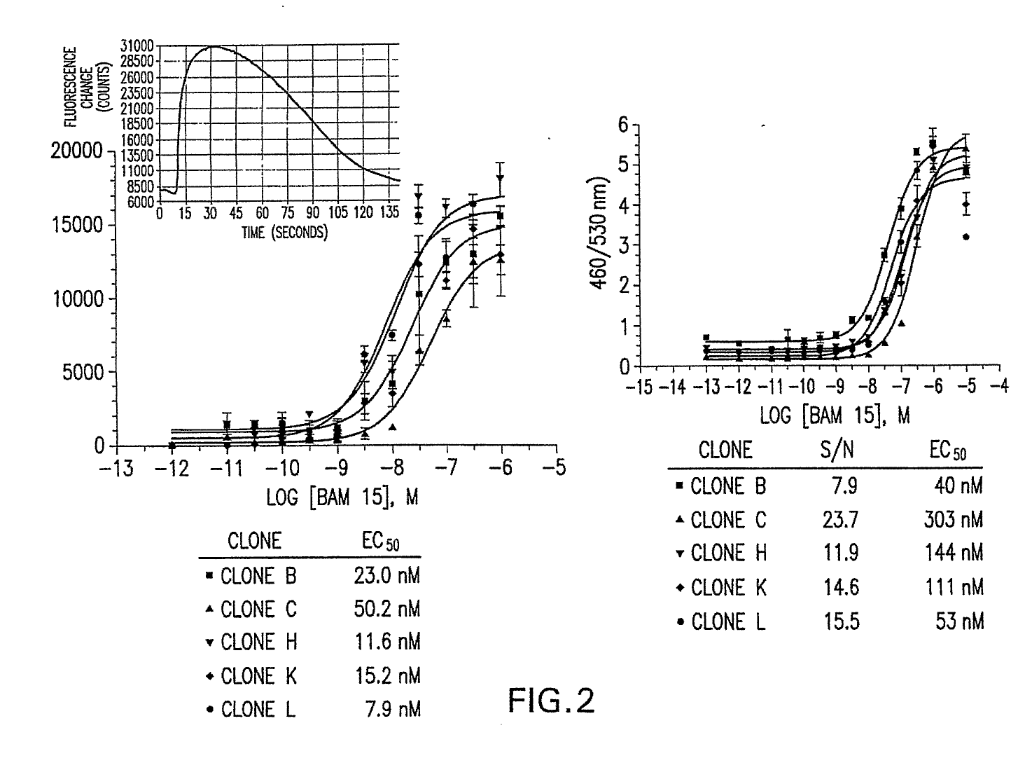

The single cell clones were grown up and analyzed for their ability to release i[Ca²⁺] upon agonist stimulation, measured by a calcium sensing probe using the fluorescence imaging plate reader (FLIPR) and the BLA assay. As assessed by FLIPR measurements (FIG. 2), the 5 clones tested appeared to have varying sensitivities to BAM15, based on their EC₅₀ response. Clones L and H appeared to be the most sensitive cell lines with the lowest EC₅₀, followed by clones B and K, respectively. Clone C appeared to be the least sensitive cell line with an EC50 of 50 nM for BAM15 (FIG. 2A). Surprisingly, the reporter-gene BLA assay was less discriminatory among these cell lines, exhibiting EC₅₀s of 40-300 nM for BAM15 (FIG. 2B). In the BLA assay, clones B and L appeared to be the most sensitive cell lines, followed by clones H and K. Similar to the second messenger assay, clone C was the least sensitive in the BLA assay. Based on these results, Clone L was chosen as the cell line for primary high throughput screening due to its sensitivity, day-to-day stability, assay robustness, and similar profiles in the BLA and i[Ca²⁺] assays.

High Throughput Screening

Cost effectiveness and automation compatibility are critical issues when screening large compound libraries of 1 million or more compounds. Due to the capability of the BLA assay to be miniaturized into a 1.8 μl assay in 3456-well plate format (13) offering substantial throughput and cost effectiveness, we chose the BLA assay as the primary assay for high throughput screening.

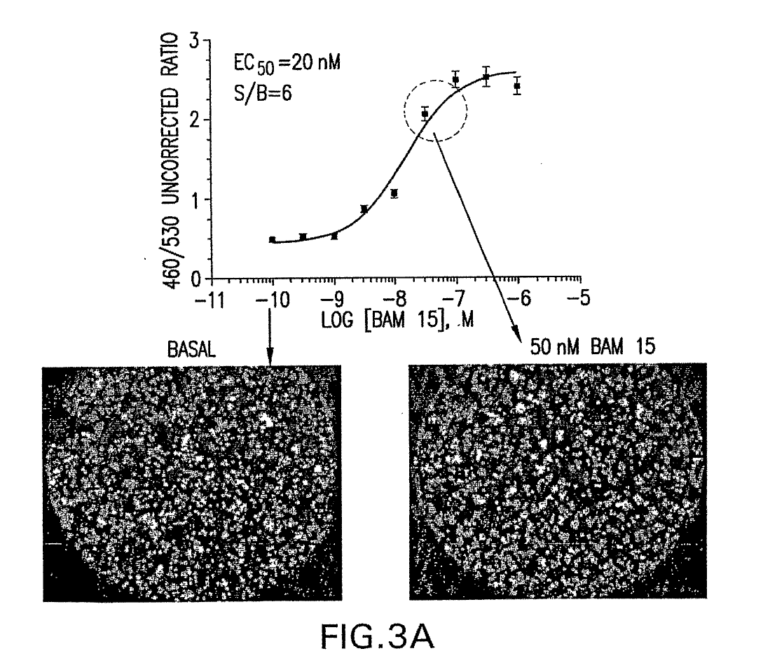

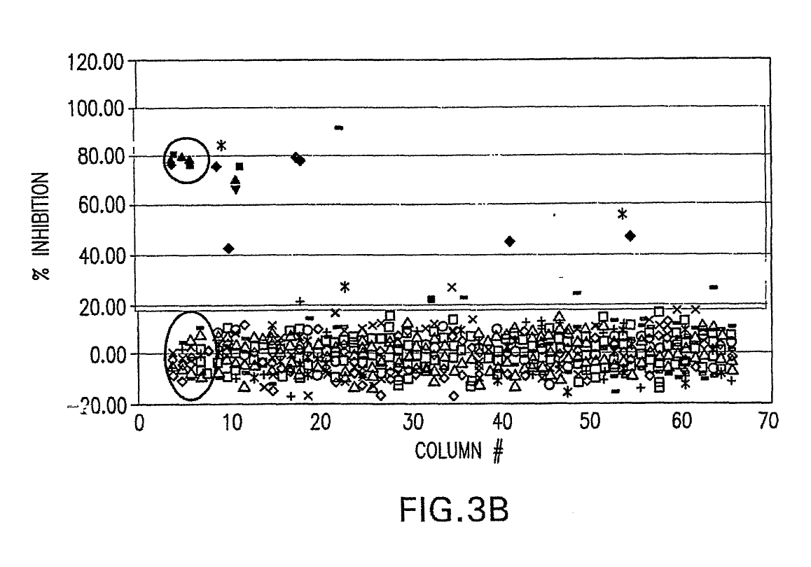

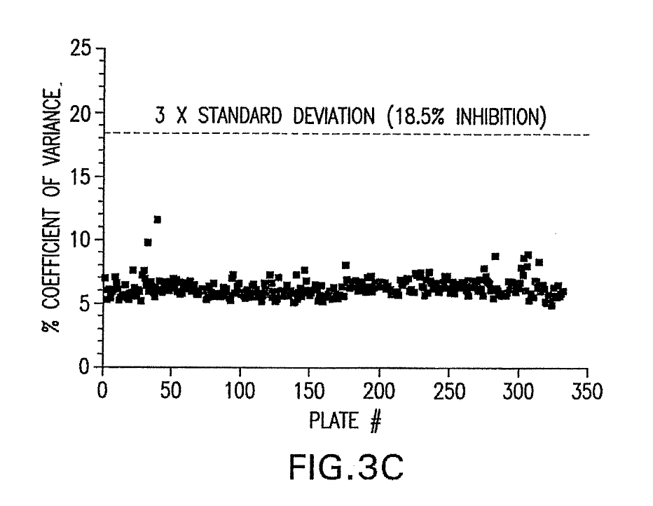

The corroboration of the receptor pharmacology in the 3456-well BLA assay is an important step in the assay validation process prior to screening, since the assay protocol in the 3456-well plates differs form conventional assays with adherent cells. In the 3456-plate format, freshly dissociated cells are used in the assay instead of plating cells 24 hours before the assay (as is customary for most adherent cell lines) in order to minimize the time-dependent evaporation effects. Furthermore, in the 3456-well screening assay, the compounds are pre-plated into assay plates (13) before the addition of live cells, unlike conventional cell-based assays, where the cells are added first to the assay plate and allowed to adhere overnight, before compound addition. The MRG-X1 BLA assay miniaturized into a 1.8 μl assay in 3456-well assay plates exhibits an EC₅₀ of 20 nM (FIG. 3A), comparable to the original 384-well assay.

Since we were interested in identifying hMRG-X1 receptor antagonists, we chose to implement the HTS assay in the presence of 50 nM BAM15 (˜EC70) against ˜1 million small molecule compounds at a final concentration of 1 μM on the Ultra High Throughput Screening System (from Aurora Discovery) (13). FIG. 3B represents a randomly chosen assay plate from the screen. Each assay plate in HTS contained 2880 wells with a unique compound pre-plated in each well, followed by the addition of the CHO-hMRGX1-NFAT-BLA cells and 50 nM BAM15. The rest of the wells in the 3456-well assay plate were used for positive and negative controls such as cells with vehicle only and cells stimulated with EC100 (500 nM) BAM15, respectively. The median response+3X standard deviation of the 2880 compound-containing wells in all such assay plates was determined to be 18.5% inhibition for the entire screen (FIG. 3C). Hence, this threshold of >20% inhibition was selected as the definition of a “primary hit”. A total of 15 such hits are present in the example assay plate shown in FIG. 3B. The shaded region between the 100% inhibition control and the median+3X standard deviation of the sample field represents the practical assay window for identifying hits.

Lead Compound Identification

The primary screen resulted in a total of 352 confirmed hits in the BLA assay. Given the long signaling pathway represented by reporter-gene assays and the potential for assay-related artifacts (11), we chose to analyze these 352 compounds in the BAM15-induced i[Ca²⁺] transient assay. This second messenger assay narrowed down the number of hits to 146 (i.e., only 146 compounds exhibited measurable inhibition of the 25 nM BAM-induced i[Ca²⁺] response). The rest 206 compounds that did not score in the i[Ca²⁺] assay may be (i) acting somewhere else in the pathway between the generation of i[Ca²⁺] and the BLA reporter, and/or (ii) may exhibit cellular toxicity resulting in a non-receptor mediated decreased BLA response (11).

Extensive structural similarity searches were conducted on the 146 confirmed compounds using a hierarchical clustering utility based on the DICE similarity of Atom-pair descriptors with a default similarity cutoff of 0.6 (16). These data mining endeavors ultimately led to the initial selection of 26 interesting compounds for further analysis on the MRG-X1 receptor.

Characterization of Lead Compounds

In order to verify that the selected compounds were indeed acting on the MRG-X1 receptor, the ligand-receptor interaction was further characterized in the CHO-hMRG-X1-NFAT-BLA cells using [³H]-BAM15. In this whole cell binding assay, the MRG-X1 receptors exhibit a Kd of 80 nM for BAM15 and a Bmax of ˜40,000 receptors/cell. The EC50 for the agonist peptide is thus comparable between the ligand binding, second messenger and reporter gene assays using the CHO-hMRG-X1-NFAT-BLA cells.

Analysis of the 26 selected compounds in the whole cell binding assay revealed that several of these compounds were indeed competitive receptor antagonists. Furthermore, structural analysis revealed one large family of 16 compounds (Table 1) with significant structure-activity relationship. Two other compounds active in the whole cell binding assay had unrelated structures (data not shown). In the large 16-member family of 2,3-disubstituted azabicyclo-octanes, the diphenylmethyl moiety appears constant at the 2-substituent, while the 3-substituent is directly correlated with the antagonist activity of the compound. The most potent member of this family is Compound 1, exhibiting and IC₅₀ of 50 nM in the BLA assay and 320 nM in the receptor binding assay.

These 26 compounds were further analyzed in a proximal, high content, receptor internalization/trafficking assay. This assay is based on the recruitment of arrestin proteins to the activated and phosphorylated receptor, resulting in uncoupling of the receptor from the G-protein (17), followed by receptor internalization via endocytosis utilizing clathrin-coated pits (18). While there are several methods to study agonist-induced GPCR trafficking, the Transfluor assay measures receptor localization indirectly by tagging the β-arrestin with GFP and does not require manipulation of the receptor sequence (19). Receptors that bind βarrestin with low affinity form transient complexes with arrestin near the plasma membrane after the formation of clathrin-coated pits, and typically exhibit the “pit” phenotype. On the other hand, receptors that bind βarrestin with high affinity form stable complexes that internalize as a unit into intracellular vesicles, exhibiting the “vesicle” phenotype (19).

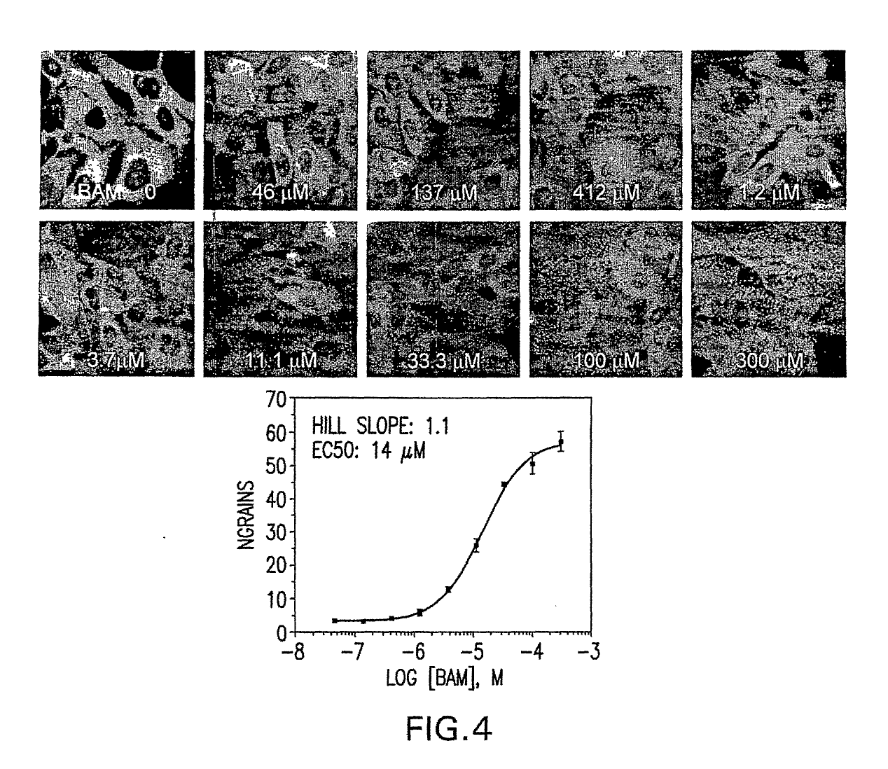

A stable hMRG-X1 cell line in U2OS cells expressing β-arrestin-GFP was developed to study the receptor internalization properties of the MRG-X1 receptor. In basal conditions, the βarrestin-GFP fluorescence (observed by the InCell 3000 confocal microscopy) appears to be located primarily in the cytoplasm, exhibiting diffuse cytoplasmic staining. This phenotype indicates that in basal conditions, there is no interaction between the βarrestin-GFP in the cytoplasm and the MRG-X1 receptors, presumably on the plasma membrane. Upon stimulation with increasing concentrations of BAM15, there is a corresponding increase in the punctuate “pit” staining within the cell, beginning from 1 μM BAM 15 and maximal at 100 μM BAM15 (FIG. 4). This corresponds to a progressive increase in the translocation of the βarrestin-GFP initially from the cytoplasm to the plasma membrane localized MRG-X1 receptors, and the subsequent endocytosis of the MRG-X1-βarrestin-GFP complex via the clathrin coated pits into intracellular pits, typically observed at later time points (30 minutes). Thus, in agonist activated cells, β-arrestin-GFP is an indirect reporter of the receptor location within the cell. The punctate β-arrestin-GFP signal within the cell upon BAM15 stimulation is indicative of activated and internalized MRG-X1 receptors existing as a low-affinity complex with βarrestin-GFP.

The punctuate or “pit” phenotype can be quantitated with appropriate algorithms, and exhibits an EC₅₀ of ˜14 μM. The reason for the rightward shift in the EC₅₀ for BAM15 in the transfluor assay compared to the FLIPR and BLA assays remains speculative at this time. It is important to bear in mind that the receptor trafficking assay is unrelated to the G protein-mediated second messenger signaling pathway (measured by the FLIPR and BLA assays), and is, instead, a direct reflection of only the βarrestin-receptor interaction. Interestingly, the lack of correlation between the agonist EC50 in the receptor trafficking assays and the second messenger assay appears to be unique for this cell line and has not been observed for other GPCRs (19; Howell, Lee and Kunapuli, personal communication).

The 26 selected compounds were further analyzed in the high content receptor trafficking assay. An advantage of this functional assay is that it has the potential to register non-competitive receptor antagonists in addition to the classical competitive antagonists identified by the receptor binding assay. Indeed, most of these compounds, including most of the non-competitive antagonists, demonstrated an ability to prevent the formation of pits in response to BAM15 in the U2OS-MRGX1-βarrGFP cells (Table I). In the presence of 10 μM concentration of each of these compounds and 60 μM BAM15 (EC80 concentration for receptor trafficking assay), the cellular phenotype appears comparable to the basal, unstimulated state, with βarrestin-GFP exhibiting diffuse cytoplasmic staining uncoupled from the receptor.

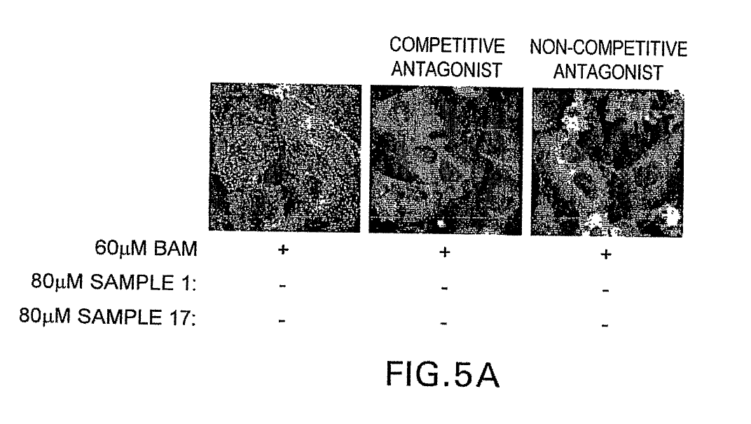

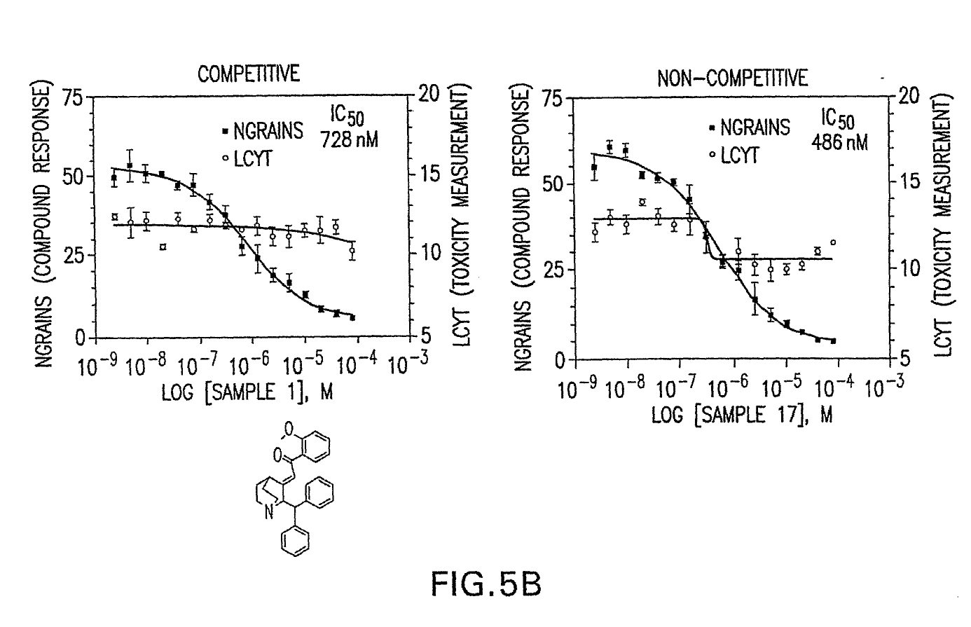

Consistent with the lower sensitivity of the receptor trafficking assay as seen with BAM15 the most potent lead, Compound 1, exhibits an IC₅₀ of 0.7 uM in the receptor trafficking assay as shown in FIG. 5. This high-content assay also reveals that Compound 1 exhibits minimal toxicity even at 100 μM, as measured by a toxicity algorithm on the InCell imaging assay. The imaging-based receptor-trafficking assay thus provides a useful method to visualize potential toxicity associated with some compounds originally identified in functional reporter-gene assays, given the potential for toxic compounds to score as “receptor antagonists” in functional assays (11).

### CONCLUSIONS

A plethora of assays were developed for the assessment of hMRG-X1 receptor antagonists, leading to the identification of several classes of hMRG-X1 receptor antagonists. The strategy used to identify these compounds reiterates the usefulness of functional cell-based assays in identifying a broader spectrum of functional antagonists. Among these functional assays, the BLA reporter-gene assay offers substantial advantages in throughput and cost that balance out the disadvantage of also obtaining compounds with off-target activities. The establishment of more proximal functional assays such as the second messenger assay and/or the receptor trafficking assay are useful to quickly eliminate the off-target activities identified originally in reporter-gene assays, while following-up on the true receptor antagonists. Overall, the receptor internalization assay provides a useful functional and proximal tool to verify the mechanism of compound action, particularly in the absence of a suitable radioligand, or, as in the case of the hMRGX1, where the radioligand binding assay may be cumbersome, with limited throughput.

Future work involves determining the specificity of these compounds for the MRGX1 receptor subtype. Overall, the identification of competitive MRGX1 receptor antagonists would prove to be useful as molecular tools to directly explore the role of these receptors in pain perception. Future work will also be focused on the delineation of the mechanism of action of some of the non-competitive compounds, and could shed light on the molecular mechanisms of receptor desensitization, in addition to being useful tools to explore the biology of the MRG family of receptors.

TABLE I
Structure-function relationship of competitive antagonists

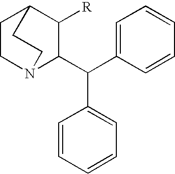

| | | IC₅₀ | | | |
|---|---|---|---|---|---|
| | | (nM) | | | |
| Compound # | R-group | BLA | FLIPR | InCell | Binding |
| 1 | 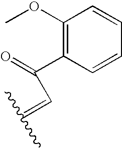 | 50 | 103 | 730 | 320 |
| 2 | 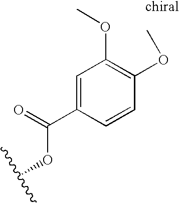 | 64 | 157 | 2800 | ND |
| 3 | 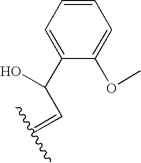 | 80 | 187 | 550 | 182 |
| 4 | 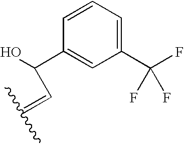 | 90 | 209 | 6782 | ND |
| 5 | 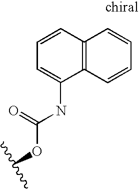 | 124 | 904 | 2200 | 1220 |
| 6 | 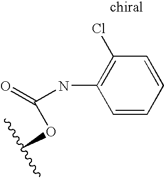 | 140 | 751 | 3600 | 607 |
| 7 | 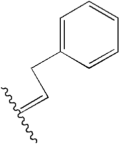 | 163 | 220 | 2200 | 1430 |
| 8 | 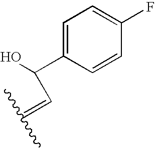 | 165 | 334 | 4800 | 630 |
| 9 | 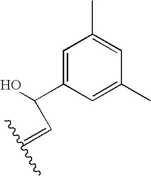 | 200 | 1500 | 5200 | ND |
| 10 | 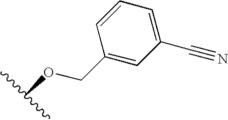 | 209 | 492 | 10000 | ND |
| 11 | 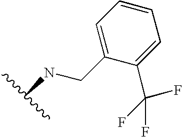 | 244 | 460 | 5200 | 1290 |
| 12 | 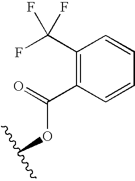 | 252 | 3300 | 8800 | 2400 |
| 13 | 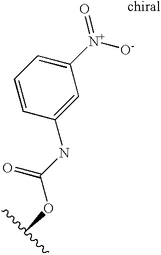 | 290 | 1300 | 6300 | 1850 |
| 14 | 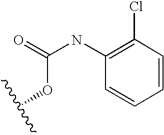 | 375 | 2700 | 29000 | 1180 |
| 15 | 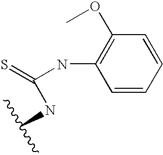 | 418 | 9800 | 13500 | 2510 |
| 16 | 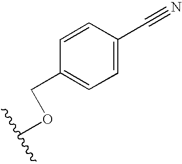 | 556 | 632 | 5600 | 1850 |

### REFERENCES

1. Drews, J. (2000) Science 287, 1960-1964.
2. Hopkins, A. L., and Groom, C. R. (2002) Nature Rev. Drug Disc. 1, 727-730.
3. Bleicher, K. H. et. al., (2003) Nature Rev. Drug Disc. 2, 369-378.
4. Civelli, O., Nothacker, H—P., Saito, Y., Wang, Z., Lin, S. H. S., Reinscheid, R. K. (2001) Trends Neurosci. 24, 230-237.
5. Marchese, A., George, S. R., Kolalowski, L. F. Jr., Lynch, K. R., O'Dowd, B. F. (1999) Trends. Pharmacol. Sci. 20, 370-375.
6. Dong, X., Han, S-K., Zylka, M. J., Simon, M. I., and Anderson, D. J. (2001) Cell 106, 619-632.
7. Lembo, P. M. C., Grazzini, E., Groblewski, T., O'Donnel, D., Roy, M-O, Zhang, J., Hoffert, C., Cao, J., Schmidt, R., Pelletier, M., Labarre, M., Gosselin, M., Fortin, Y., Banville, D., Shen, S. H., Strom, P., Payza, K., Dray, A., Walker, P., and Ahmad, S. (2002) Nature Neoroscience 5, 201-209.
8. Boersma, C. J., Pool, C. W., Van Heerikhuize, J. J., and Van Leeuwen, F. W. (1994) J. Neuroendrocrinol. 6, 47-56.
9. Quirion, R., and Weiss, A. S. (1983) Peptides 4, 445-449.
10. Rao, A., Luo, C., Hogan. P. C. (1997) Annu. Rev. Immunol. 15, 707-747.
11. Kunapuli, P., Ransom, R., Murphy, K. L., Pettibone, D., Kerby, J., Grimwood, S., Zuck, P., Hodder, P., Lacson, R., Hoffman, I, Inglese, J., Strulovici, B. (2003) Anal. Biochem. 314, 16-29.
12. Zlokarnik, G., Negulescu, P., Knapp, T. E., Mere, L., Burres, N., Feng, L., Whitney, M., Roemer, K., Tsein, R. Y. (1998) Science 279, 84-88.
13. Komienko, O., Lacson, R., Kunapuli, P., Schneeweis, J., Hoffman, I., Smith, T., Alberts, M., Inglese, J., and Strulovici, B. (2004) J. Biomol. Screening. 9, 186-195.
14. Hodder, P., Cassaday, J., Peltier, R., Berry, Ku., Inglese, J., Feuston, B., Culberson, C., Bleicher, L., Cosford, N. D. P., Bayly, C., Suto, C., Varney, M., Strulovici, B. (2003) Anal. Biochem. 313, 246-254.
15. Hodder, P; Mull, R; Cassaday, J; Berry, K; Strulovici, B. (2004) J. Biomol. Screening. 9, 417-426.
16. Kearsley, S. K., Sallamack, S., Fluder, E. M., Andose, J. D., Mosley, R. T., Sheridan, R. P. (1996) J. Chem. Inf. Comput. Sci. 36, 118-127.
17. Lefkowitz, R. J. (1998) J. Biol. Chem. 273, 18677-18680.
18. Goodman, O. B. Jr., Krupnick, J. G., Santini, F., Gurevich, V. V., Penn, R. B., Gagnon, A. W., Keen, J. H., and Benovic, J. L. (1996) Nature 383, 447-450
19. Oakley, R. H., Hudson, C. C., Cruickshank, R. D., Meyers, D. M., Payne, R. E., Rhem, S. M., Loomis, C. R. (2002) Assay Drug Devel. Tech. 1, 21-30.

What is claimed is:

1. A method for treating a disease or condition mediated by the human MRG-X1 receptor in a human patient in need thereof comprising administering to the patient a therapeutically effective amount of a compound of Formula I 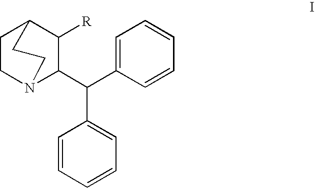
or a pharmaceutically acceptable salt thereof, wherein:
R is 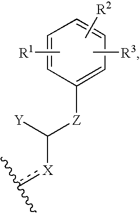
wherein: [symbol]
represents an optional double bond;
when [symbol] is a single bond, X is selected from the group consisting of: —O—, —S—, —NH— and —CH₂—;
when [symbol] is a double bond, X is selected from the group consisting of: ═N— and ═CH—;
Y is selected from the group consisting of: H, —OH, ═O, ═S and halo;
Z is selected from the group consisting of: a bond, —O—, —S—, —NH— and —CH₂—;
R¹, R² and R³ are independently selected from the group consisting of: H, C₁₋₆alkyl, C₂₋₆alkenyl, C₂₋₆alkynyl, halo, cyano, nitro, trifluoromethyl, trimethylsilyl, —OR^{a}, SR^{a}, SOR^{a}, SO₂R^{a}, —NR^{a}R^{b}, —NR^{a}COR^{b}, —NR^{a}CO₂R^{b}, —CO₂R^{a} and —CONR^{a}R^{b},
and any two of R¹, R² or R³ may be joined together with the phenyl atom to which they are attached to form naphthyl; and
R^{a} and R^{b} are independently selected from the group consisting of: H, C₁₋₆alkyl, phenyl and trifluoromethyl.

2. The method according to claim 1 wherein Z is —NH—.

3. The method according to claim 1 wherein Z is a bond.

4. The method according to claim 1 wherein [symbol] represents a double bond.

5. The method according to claim 1 wherein [symbol] represents a single bond.

6. The method according to claim 1 wherein X is O.

7. The method according to claim 1 wherein Y is OH.

8. The method according to claim 1 wherein Y is ═O.

9. The method according to claim 1 wherein R is selected from the following table: 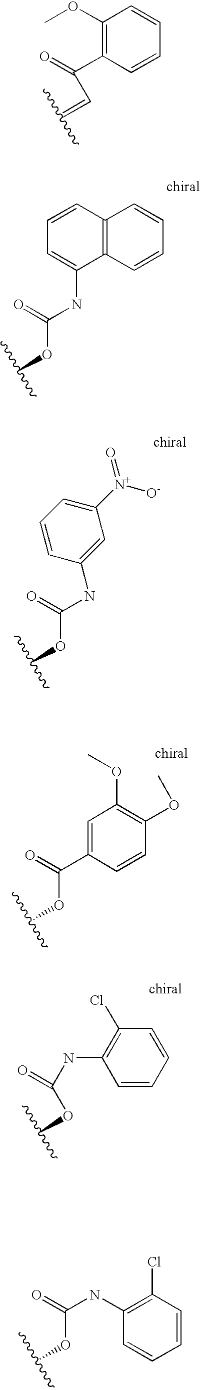 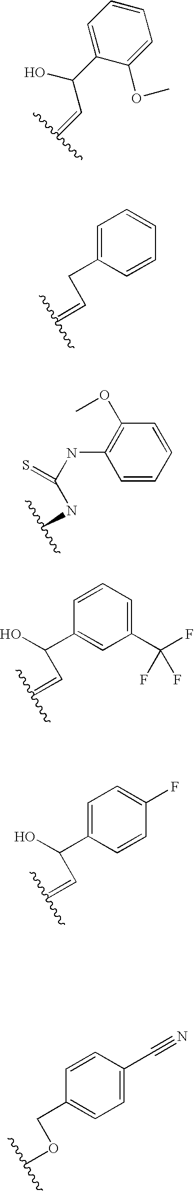
or a pharmaceutically acceptable salt of any of the above compounds.

10. The method according to claim 1 wherein the disease or condition mediated by the human MRG-X1 receptor is selected from the group consisting of: nociception, hyperalgesia, allodynia, pain related to central hypersensitivity conditions, somatic pain, visceral pain, acute pain, chronic pain, post-operative pain, headache, inflammatory pain, neurological pain, musculoskeletal pain, cancer related pain and vascular pain.

11. A method for assessing the potency of a candidate compound that is an antagonist of the MRG-X1 receptor comprising:
(1) determining the potency of the candidate compound in a MRG-X1 receptor binding assay;
(2) determining the potency of a compound of Formula I 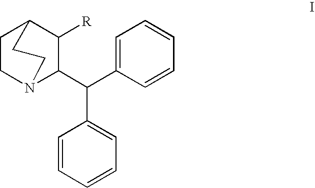
or a pharmaceutically acceptable salt thereof, wherein:
R is 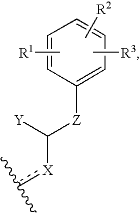
wherein: [symbol]
represents an optional double bond;
when [symbol] is a single bond, X is selected from the group consisting of: —O—, —S—, —NH— and —CH₂—;
when [symbol] is a double bond, X is selected from the group consisting of: —N— and ═CH—;
Y is selected from the group consisting of: H, —OH, ═O, ═S and halo;
Z is selected from the group consisting of: a bond, —O—, —S—, —NH— and —CH₂—;
R¹, R² and R³ are independently selected from the group consisting of: H, C₁₋₆alkyl, C₂₋₆alkenyl, C₂₋₆alkynyl, halo, cyano, nitro, trifluoromethyl, trimethylsilyl, —OR^{a}, SR^{a}, SOR^{a}, SO₂R^{a}, —NR^{a}R^{b}, —NR^{a}COR^{b}, —NR^{a}CO₂R^{b}, —CO₂R^{a} and —CONR^{a}R^{b},
and any two of R¹, R² or R³ may be joined together with the phenyl atom to which they are attached to form naphthyl; and
R^{a} and R^{b} are independently selected from the group consisting of: H, C₁₋₆alkyl, phenyl and trifluoromethyl, in a MRG-X1 receptor binding assay; and
(3) comparing the potency of the candidate compound to the potency of the compound of Formula I to determine if the candidate is more or less potent than the compound of Formula I.

12. A method for validating the MRG-X1 receptor to treat a disease or condition believed to be mediated by the MRG-X1 receptor, comprising administering an effective amount of a compound of Formula I 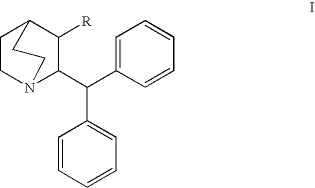
or a pharmaceutically acceptable salt thereof, wherein:
R is 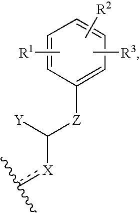
wherein: [symbol]
represents an optional double bond;
when [symbol] is a single bond, X is selected from the group consisting of: —O—, —S—, —NH— and —CH₂—;
when [symbol] is a double bond, X is selected from the group consisting of: —N— and ═CH—;
Y is selected from the group consisting of: H, —OH, ═O, ═S and halo;
Z is selected from the group consisting of: a bond, —, —S—, —NH— and —CH₂—;
R¹, R² and R³ are independently selected from the group consisting of: H, C₁₋₆alkyl, C₂₋₆alkenyl, C₂₋₆alkynyl, halo, cyano, nitro, trifluoromethyl, trimethylsilyl, —OR^{a}, SR^{a}, SOR^{a}, SO₂R^{a}, —NR^{a}R^{b}, —NR^{a}COR^{b}, —NR^{a}CO₂R^{b}, —CO₂R^{a} and —CONR^{a}R^{b},
and any two of R¹, R² or R³ may be joined together with the phenyl atom to which they are attached to form naphthyl; and
R^{a} and R^{b} are independently selected from the group consisting of: H, C₁₋₆alkyl, phenyl and trifluoromethyl, to an animal in an in vivo model to test whether the compound is useful to treat the disease or condition

13. The method according to claim 12 wherein the disease or condition is pain.

14. The use of a compound of Formula I 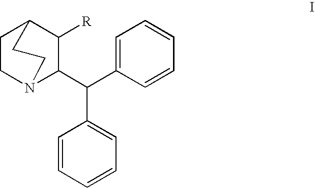
or a pharmaceutically acceptable salt thereof, wherein:
R is 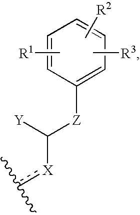
wherein: [symbol]
represents an optional double bond;
when [symbol] is a single bond, X is selected from the group consisting of: —O—, —S—, —NH— and —CH₂—;
when [symbol] is a double bond, X is selected from the group consisting of: ═N— and ═CH—;
Y is selected from the group consisting of: H, —OH, ═O, ═S and halo;
Z is selected from the group consisting of: a bond, —O—, —S—, —NH— and —CH₂—;
R¹, R² and R³ are independently selected from the group consisting of: H, C₁₋₆alkyl, C₂₋₆alkenyl, C₂₋₆alkynyl, halo, cyano, nitro, trifluoromethyl, trimethylsilyl, —OR^{a}, SR^{a}, SOR^{a}, SO₂R^{a}, —NR^{a}R^{b}, —NR^{a}COR^{b}, —NR^{a}CO₂R^{b}, —CO₂R^{a} and —CONR^{a}R^{b},
and any two of R¹, R² or R³ may be joined together with the phenyl atom to which they are attached to form naphthyl; and
R^{a} and R^{b} are independently selected from the group consisting of: H, C₁₋₆alkyl, phenyl and trifluoromethyl, to bind to the MRG-X1 receptor in an in vitro or in vivo assay, test or model.
